(19) United States
(12) Patent Application Publication
(10) Pub. No.: US 2012/0259025 A1
(43) Pub. Date: Oct. 11, 2012

(54) Method and Systems Thereof of Ecologically Carbon Dioxide-Neutral Methanation

(76) Inventor: Qingchun Zhao, Longmont, CO (US)

(21) Appl. No.: 13/369,501

(22) Filed: Feb. 9, 2012

Related U.S. Application Data
(60) Provisional application No. 61/473,935, filed on Apr. 11, 2011.

(51) Int. Cl. C07C 27/00; B01J 19/00

(52) U.S. Cl. 518/704; 422/162

(57) ABSTRACT

The invention presented herein is an integrated method and the systems based on the said method, which utilize renewable energy, or any energy the production of which emits little or no carbon dioxide (CO₂), or the syngas produced from the said method, for generating electricity that powers the said systems; convert CO₂ collected from either the open air or any CO₂ emission sites into methane by reacting CO₂ with hydrogen produced with no or little CO₂ emission; store or transport methane or natural gas making use of the existing natural gas distribution system; providing a novel approach of using and expanding the usage of renewable energy; providing an ecologically CO₂-neutral natural gas resource which allows human society to keep being driven by hydrocarbon fuels while not worrying about global warming.

### CROSS-REFERENCE TO RELATED APPLICATIONS

This application claims the benefit of U.S. Provisional Application No. 61/473,935, filed Apr. 11, 2011, which are incorporated herein by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### FIELD OF THE INVENTION

The invention presented herein relates to renewable energy utilization, combat against global warming and innovation of novel natural gas resources.

### BACKGROUND OF THE INVENTION

To date the issues that are considered to severely affect the earth planet are both energy-related and environmental. The demand of human society for energy has been increasing more than ever. In addition to the traditional fossile fuels, human are making all the efforts in finding new energy resources. However, with the current capability of collecting renewable energy, it is perceived that we will continue relying heavily on fossil fuels (oil, natural gas, etc.) for driving human activities. Burning carbon generates 75% of the total electricity in the world. Through the year 2035 coal and natural gas will keep contributing over 60% of the fuel burning generated electricity. Natural gas is a widely used energy resource for power generation, industrial and residential use. Fossil fuels and natural gas are mostly from mineral exploration worldwide. There are concerns about energy resources sustainability in the future. Meanwhile, burning fossil fuel brings up environmental problems. Fossil fuel burning power plants are contributing over 40% of carbon dioxide (CO₂) anthropogenically emitted into the atmosphere. CO₂ emitted by human activity is considered the major component of green house gases that is blamed for causing global warming.

Another fact is that human have developed renewable energy resources, such as wind, solar, hydro, tide wave, nuclear power and some innovative ideas in the R&D stage that do not emit as much CO₂ as fuel burning power plants do, to slow down global warming and hopefully to stop it ultimately. The problem with these renewable resources is reliability. For instance, solar energy can be harvested when the sun shines; during night time it cannot be generated. Wind power is only available when wind is mild. When there is no wind the power cannot be generated; when wind is too strong, currently the wind power has to be protectively shut down. The wind energy hasn't been collected effectively. The question then is what we can use as a power source when the unreliable renewable energy is not available, which would emit no or least CO₂ into the open air. The current solution is energy storage. The renewable energy is stored when it is available and released for use when it cannot be generated from natural sources. A typical device for this purpose is a fuel cell which is formed in many ways. Currently energy storage is mostly for providing electricity when the renewable energy cannot be generated. On the other hand, the use of renewable energy can be limited by the power grid capacity. Generally speaking, the usage of renewable energy is limited by its own reliability and the power grid capacity.

One more fact is that methane, mostly in the form of natural gas (methane mixed with a small portion of hydrogen (H₂)), has long been an energy resource. Even though the renewable energy and the stored renewable energy can provide electricity aiming at reducing the use of fuel burning that emits huge amount of CO₂ into the atmosphere in electricity generation, natural gas and other carbon-containing fuels are still being burned anywhere and anytime they need to be burned for generating power in the forms other than electricity. For instance, burning natural gas for heating, burning gas in vehicles, etc. That being said, the renewable energy and its storage, when it gets the fuel-burning power plants all replaced, can at most eliminate 40% of CO₂ emitted by human activities, the share of fuel-burning power plants in CO₂ emission from human activities. Meanwhile, in any places (transportation, industrial, residential) where natural gas and other fuels have to be burned CO₂ continuously emits, which counts for the other 60% of total CO₂ emitted due to human activities. Reduction and elimination of this 60% of CO₂ emission hasn't been addressed effectively. Although electric cars have already started to run on the roads, their problem at root is the difficulties in effective energy storage.

Therefore, a solution is sought that can enable three things together: 1) reliably and more broadly using renewable energy; 2) reducing and eliminating CO₂ emission from not only the fuel-burning electricity generation but also other categories (transportation, industry and residential); and 3) providing a new natural gas source that could supplement the foreseen natural gas shortage in the future and potentially replace the mine sources of both natural gas and fuel. It is exactly what the present invention is for. The invention herein provides a solution that takes in CO₂ that otherwise emits into the atmosphere, makes renewable energy into methane that is then stored into public natural gas system. Consumed in the form of methane or natural gas and taking advantage of the existing public natural gas transportation/storage system, the utilization of renewable energy becomes practically reliable and broadened. Meanwhile, human can just collect as much renewable energy as they can with the existing natural gas storage system. Literally there is no limit of collecting renewable energy in this sense. A new natural gas source as such not only is ecologically CO₂ neutral in the atmosphere but also can supplement or replace the mine sources of natural gas and fossil fuels. It addresses the issue of renewable energy reliability; it addresses the concern on the shortage of mined energy sources in the future; it addresses the issue of stopping global warming. In addition, the invented solution is benefited from oxygen generation as a positive side product for human use.

### BRIEF SUMMARY OF THE INVENTION

This invention provides an integrated method that uses renewable energy, converts CO₂, which is collected from either the atmosphere or any sites where CO₂ is produced as side effect and otherwise could emit into the atmosphere, into methane, distributes the said methane with or without hydrogen through the existing natural gas distribution systems nationwide. From the perspective of renewable energy utilization, the said method uses CO₂ as working medium, converts the renewable energy into methane other than electricity or hydrogen. It broadens the ways of utilizing renewable energy beyond electricity. Consuming renewable energy in the form of methane can take advantage of the existing public natural gas transportation and storage system, relieving the current and inherent difficulties in renewable energy storage and usage. From the perspective of exploring new energy resources, the said methane can be consumed directly or further converted into other fuels which can be consumed in place of those that are supplied from the currently existing sources of fuels and natural gas. Since burning the fuels produced as such does not produce CO₂ more than the CO₂ feedstock in the invented method, the said method actually provides a new fuel resource that is ecologically CO₂ neutral. From the perspective of combating global warming, obviously the said method presents an effective way of depressing the CO₂ level in the atmosphere.

The invented method involves two major chemical reactions: one is to perform water electrolysis producing hydrogen with oxygen as by-product; another is Sabatier reaction that reacts CO₂ and hydrogen to produce methane and water. All the inputs and outputs of the said method are designed facilitating these two reactions. The invented method is powered with renewable energy, or more generally, any energy the generation of which emits little or no CO₂ into atmosphere. The said renewable energy is used for generating electricity and/or heat. The generated electricity provides power for system operations; the optionally generated heat assists in chemical reactions. Water and CO₂ are must-have feedstock. Optionally external hydrogen source is provided in case that hydrogen production in the said method is inadequate supplying for Sabatier reaction. Heat source is optionally provided assisting water electrolysis and CO₂ feed-in. An alternator is provided to optionally convert the reaction heat into electricity. The heat produced during the whole process in the said method is recycled and reused for assisting water electrolysis, and/or CO₂ collection, and/or electrical power supply. The Sabatier reaction residuals—water, CO₂, hydrogen and some methane—are recycled and reused. The produced methane is output, with or without small amount of hydrogen, to the existing natural gas system or put into storage. Oxygen, the byproduct of water electrolysis, is also output for storage or commercial use.

The invented method uses CO₂ which is extracted from the atmosphere or otherwise emitted into the atmosphere. Therefore the CO₂ sources include the open air and all kinds of CO₂ emitting sites (fuel burning sites, cement factories, refineries, vehicles and ships, etc.) covering the sectors of industry, energy, transportation and residential.

Depending on the scale of practical application need, a realistic system can be configured with different components upon the said method. Depending on the realistic system configuration, the system efficiency arranges through 10% to 95% or up; the synthesized gas output is either methane itself or a mixture of methane and small amount of hydrogen, exactly like natural gas.

The invented integrated systems in line with the invented method comprise 1) generation of electricity with renewable energy, or methane which is produced according to the said method, 2) heat source, 3) water source, 4) an alternator, 5) water electrolyzer, 6) Hydrogen source, 7) CO₂ source, 8) Sabatier reactor, 9) gas separator, 10) a variety of conduits transferring electricity, heat and gases, 11) oxygen storage, 12) storage of the mixture of methane and hydrogen, 13) control system. Each component has multiple sub-system-level configuration options upon application needs. The invented integrated systems can be in different scale depending on application needs.

The said renewable energy can be, but is not limited to, wind power, solar photovoltaic (PV), hydro powers, tides. As long as it does not emit or emit little CO₂, any energy source can be used functionally as the renewable energy. The electricity can also be generated by consuming the methane produced by the herein invented method and systems.

The heat source is provided for assisting water electrolysis and/or CO₂ feed-in. Heat source can be, but is not limited to, a device/instrument/apparatus/equipment that collects radiance from the sun or any glowing object, which can be, but not limited to, electrical heater and/or combustion, and/or that collects heat from mechanical friction.

The water source can be any water storage on-site, including tanks, lakes, etc. Water is filtered before fed into the water electrolyzer.

The alternator is optionally used in one preferred embodiment in which heat management reuses the heat from Sabatier reaction, partially or fully, converting it in any ways to electricity.

The H₂ source is supplemental in case on-line hydrogen production is insufficient. The hydrogen supplied by the H₂ source should be produced with any methods that generate none or little of CO₂ emission.

The CO₂ source is any device/instrument/apparatus/equipment that takes in and/or stores CO₂ either from the open air or from a CO₂ emitting source in the sectors of industry, energy, transportation and residential, which includes, but not limited to, coal-fired power plants, refineries, cement factories, vehicles, ships, residential utility, etc.

Sabatier reactor conducts the reaction of 4H₂+CO₂ [symbol] CH₄+2H₂O. It is assisted with catalysts (Nickel, Rhuthium, etc.) and of a couple of types: microchannel, single-tube, flat-bed, etc. with no preference. They have different efficiencies ranging from 50% to 99%.

Gas separator is associated with the Sabatier reactor to isolate methane, hydrogen, CO₂ and water. It can be any device/instrument/apparatus/equipment with any technologies that are used in industry and known to those skilled in the art. It can allow some hydrogen output together with methane, a syngas mimicking the natural gas. The performance of the gas separator plays an important role in determining the system-level efficiency.

Sabatier reaction is very exothermic generating a lot of heat. Recycling and reusing this heat improves the system efficiency. Hence heat management is included in the invented systems. Aside of the heat management, effective recycling and reusing the reaction products of water, CO₂, hydrogen and their mixture helps in improving the system efficiency as well. Thus they are managed in the invented systems, too. These managements are implemented with a variety of conduits and tubes.

The reaction product of oxygen is stored. It can be utilized in other applications benefitting the invented systems.

The reaction product of methane, or the mixture of methane and hydrogen, is transferred to the existing natural gas system for commercial consumption or stored for later use generating electricity for the invented system.

### BRIEF DESCRIPTION OF THE DRAWINGS

FIG. 1: The system-level schematic of the ecologically CO₂ neutral methanation system. It lays out the general configuration of the invented system.

FIG. 2: CO₂ sources. It enumerates the sectors of CO₂ sources.

FIG. 3: Usage of the produced methane/natural gas. It classifies how the produced methane/natural gas is consumed.

FIG. 4: Novel vehicle equipped with exhaust tank and novel gas station equipped with exhaust recycling facility. It illustrates a novel concept of collecting CO₂ from vehicles.

FIG. 5: Ecologically CO₂ neutral fuel production/consumption cycle. It illustrates the complete cycle of CO₂-neutral ecology with the invented method/system playing the role of CO₂-to-methane converter.

### DETAILED DESCRIPTION OF THE INVENTION

Methodologically the presented invention uses renewable energy of any types, or in another term, any energy the generation of which does not at all or least emits CO₂ into the atmosphere; takes in CO₂ that otherwise would stay in the atmosphere; produces hydrogen with the renewable energy through water electrolysis where the oxygen is produced as well; converts hydrogen and CO₂ into methane and water; has methane and realistically some hydrogen stored and transferred for use as natural gas; and has the heat, the reaction residual of hydrogen, water and CO₂ as well as the unseparated mixture of them and slight amount of methane recycled and reused. A system can be built at various scales with different configurations for a variety of applications.

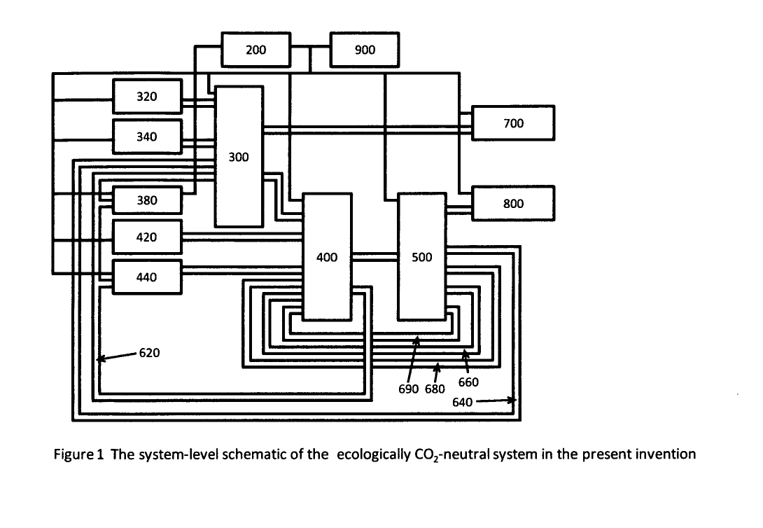

FIG. 1 demonstrates the composition of the invented integrated method. In FIG. 1 the individual component is numbered and represented with solid-lined rectangular box framing its number. Single line represents power distribution. The electricity generated from the renewable energy is supplied to all the components. Double line represents conduits and pipes that recycle heat, reaction residuals of water, hydrogen, methane and CO₂. The whole heat and residual gas management is numbered 600 which is not shown in FIG. 1 but composed of 620, 640, 660, 680 and 690. There are also wires connecting the control unit with all other components, which are not shown in FIG. 1 but known to those who are skilled in the art. The main stream of the whole process follows. The renewable energy converted electricity (200) provides power for operating the system; hydrogen is generated through the water electrolyzer (300), which generates oxygen for other use/storage (700). If additional hydrogen is needed, the external hydrogen source (420) may be used. Water electrolyzer (300) is supplied with heat from the external heat source (320) and heat recycling (620) from Sabatier reactor (400) and water from the external source (340) and water recycling (640) from gas separator (500); CO₂ is supplied through the CO₂ source (440); CO₂ from the CO₂ source (440) and recycled (660) from gas separator (500) and hydrogen from the water electrolyzer (300) and recycled from the gas separator (500) are reacted in the Sabatier reactor (400) into methane and water at an efficiency ranging from 50% up to 99%; the gas mixture of CO₂, hydrogen, water and methane is separated through gas separator (500), where water (640) is fed back to the water electrolyzer (300), CO₂ (660) and hydrogen (680) are fed back to the Sabatier reactor (400), the gas mixture (690) that is left after separation process is fed back to Sabatier reactor (400), methane with/without small amount of hydrogen are output, as product, for use/storage (800). The whole system is operated by the control unit (900) which is not drawn in detail but known to those skilled in the art. The heat generated by the exothermic Sabatier reaction (620) can be supplied to water electrolyzer (300) and/or CO₂ source (440) and/or optionally be fed to an alternator (380) generating electricity for use, preferably being supplied to the renewable energy generated electricity (200). In FIG. 1 each component is drawn within one rectangular box. However, one box represents one or more component units.

The invented method implements a sequential process. Hence the system efficiency of utilizing the renewable energy is the multiplication of the component-level efficiencies. The component-level efficiencies include power consumption for system operation, the efficiency of the water electrolyzer (300), the efficiency of the Sabatier reactor (400), the efficiency of the gas separator (500), the efficiency of recycle and reuse management (600). The system operation power consumption further includes power consumed for maintaining operation of all the components, feedstock sources and product gas storages (700) and (800). It is the overhead in energy consumption and can be complemented by harvesting more renewable energy. Essentially the efficiencies that play more important roles are of the water electrolyzer (300), Sabatier reactor (400), gas separator (500) and the recycle and reuse management (600). So from hereon we refer the system efficiency to the multiplication of the efficiencies of water electrolyzer (300), Sabatier reactor (400), gas separator (500) and the recycle and reuse management (600).

The efficiency of water electrolyzer (300) ranges from 30% up to 99% depending the technology and configuration adopted. The typical efficiency is 85%.

The efficiency of Sabatier reactor (400) can be up to 99%. The typical efficiency is 90%.

The efficiency of gas separator (500) mainly indicates the capability of isolating methane and some of hydrogen out of the gas mixture for output. It ranges from 10% up to 99% depending on specific technologies applied. The efficiencies of water electrolyzer (300), Sabatier reactor (400) and the separator (500) together determine the production rate. In some embodiments Sabatier reactor (400) comes with the gas separator (500) and only one combinational efficiency is specified.

The efficiency of the recycle and reuse management (600) plays an important role in optimizing the system efficiency. There are two efficiencies: gas (640, 660, 680 and 690) recycle and reuse efficiency and heat recycle and reuse (620) efficiency. The separated CO₂ (660), hydrogen (680) and the unseparated mixture of CO₂, hydrogen, methane and water (690) are recycled and fed back to the Sabatier reactor (400). The separated water (640) is recycled and fed back to water electrolyzer (300). If all the residual gases are recycled and fed into relevant components (number 300 and 400), 100% efficiency of gas recycle and reuse is achieved. In practice the gas recycle and reuse efficiency is less than 100%. In the present invented method/systems, the gas recycle and reuse efficiency ranges from 10% up to 99%. In a preferred embodiment with a set of efficiencies of the water electrolyzer (300), the Sabatier reactor (400), the gas separator (500) and the gas recycle and reuse, the electricity supply from the renewable energy (200) and the external CO₂ source (440) and optionally the external hydrogen source (420) can be adjusted to balance the feedstock to the Sabatier reactor (400) such that the amount of CO₂ and hydrogen equivalent to their fresh input (opposite to the recycled) could be optimally converted to methane that comes out to the storage (800). The heat recycle and reuse efficiency indicates how much heat is recycled and reused. The heat from the exothermic Sabatier reaction is taken away with the cooling water that circulates around and through the Sabatier reactor (400). In a preferred embodiment water electrolyzer (400) needs to be operated under high temperatures and/or high pressures, the circulating cooling water can be used directly aside with the external water source (340) in water electrolysis reaction, which is not explicitly shown in FIG. 1 but can be easily understood by those skilled in the art, or for warming up the water from the water source (340). In another preferred embodiment, the circulating cooling water (620) can be steamed and supplied to the alternator (380) generating additional electricity to the renewable energy generated electricity (200), hence increasing the system efficiency. In another preferred embodiment, heat is needed in collecting and/or purifying CO₂, the heat (620) from exothermic Sabatier reaction can also be fed to the CO₂ source (440) for improving system efficiency. In another preferred embodiment, the recycled heat (620) can be used for any combination of assisting water electrolysis (300), generating electricity (380) and assisting CO₂ feeding (440). In the present invented systems, the heat recycle and reuse efficiency ranges from 10% up to 99% depending on the specific system configuration and technologies adopted.

Upon different system configurations for integration, the system efficiency ranges from 10% to 95%.

As mentioned above, the renewable energy (200) means any energy the generation or harvest of it does not at all or least emits CO₂ into the atmosphere. It includes, but not limited to, wind, solar, hydro and tides or the combination of any of them.

The external heat source (320) is optional and only needed in the case that water electrolyzer (300) operates at high temperature. It can be the radiation collected from any glowing objects, or combustion of any fuels with no CO₂ emitted, or from any mechanical friction.

The external water source (340) is provided in many ways as those skilled in the art know of The provided water needs to be purified such that it qualifies for Sabatier reaction.

The alternator (380) is preferably a steam turbine electricity generator.

As mentioned above, the external hydrogen source (420) is put in use in the case that the hydrogen produced through the water electrolyzer (300) is insufficient supporting the system operation. Preferably it is produced with no or little of CO₂ emission.

As mentioned above, the external CO₂ source (440) is actually a CO₂ extractor plus storage. It absorbs CO₂ from the open air and stores it together with the CO₂ that is collected, transported in from other CO₂-collecting locations. It feeds CO₂ directly to the Sabatier reactor (400) during system operation.

The control unit (900) is a set of control and sensor system that operates the invented system. It provides functions that are needed in control and operation at all levels from the system to components as known to those skilled in the art.

The configuration of invented systems can be at various scales upon application needs. The invented systems can be as small as for supporting a single family use or as big as for supporting a region.

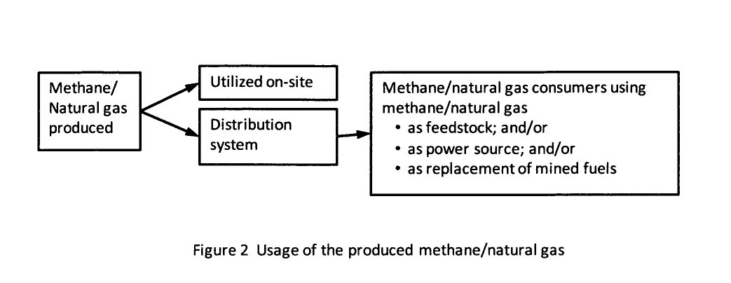

FIG. 2 demonstrates the usage of produced methane/synthesized natural gas through the presented invention. It can be consumed on-site and/or distributed via the existing natural gas transportation system to more consumers and consumed as feedstock for other products and/or power source and/or replacement of mined fuels.

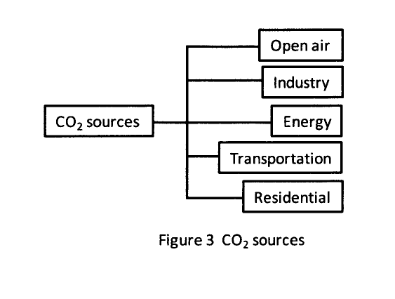

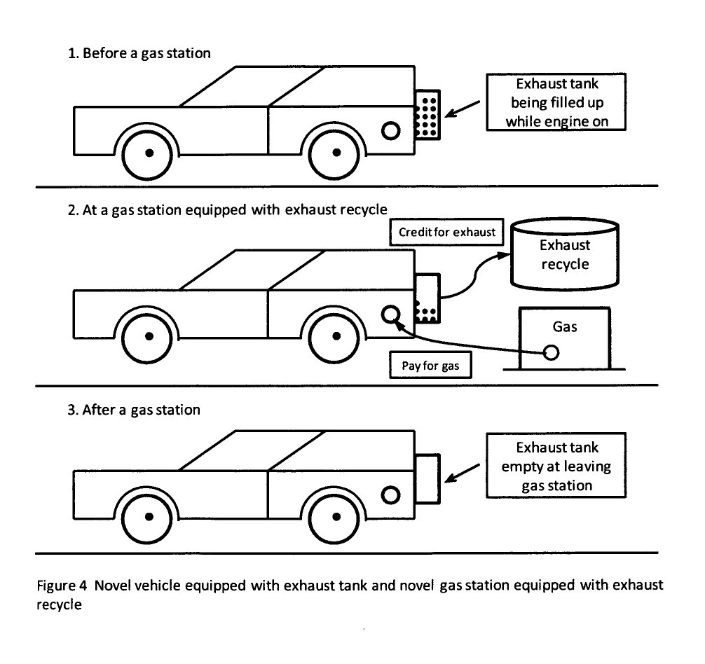

FIG. 3 shows a list of the sources of CO₂. Essentially the invented method/systems accept CO₂ that is collected from any sources. CO₂ can be collected from industry sector, energy sector, transportation and residential area. To collect CO₂, what is needed is CO₂ recycle facilities. FIG. 4 illustrates a novel concept of collecting CO₂ from vehicle exhaust which consists of a novel vehicle design and novel gas station design. The novel vehicle is equipped with an exhaust tank which collects the exhaust while the engine is running and can be emptied at the novel gas station which does not only sell gas but also recycles the exhaust.

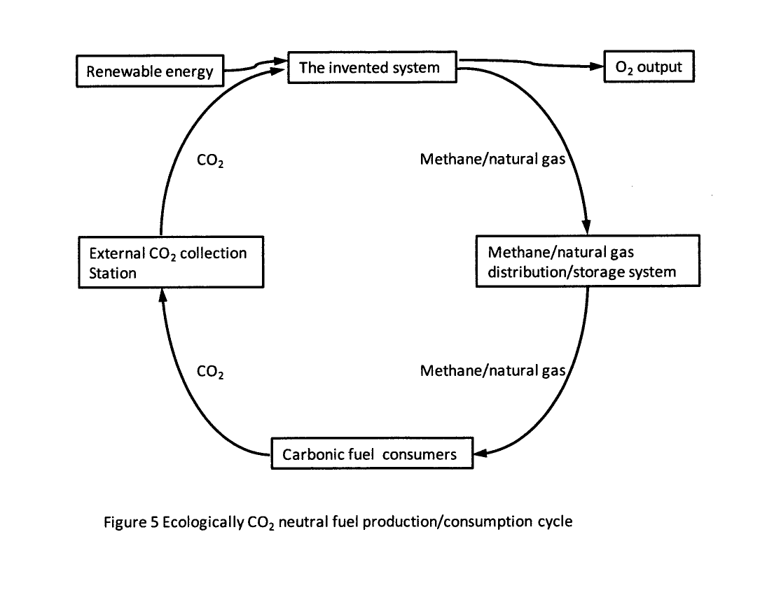

FIG. 5 illustrates the ecologically CO₂ neutral fuel production/consumption cycle which is sustained by renewable energy and in which the herein invented method/systems plays the key role of converting CO₂ into methane/natural gas.

### CLOSURE

While the invention has been disclosed in its preferred forms, it will be apparent to those skilled in the art that many modifications, additions, and deletions can be made therein without departing from the spirit and scope of the invention and its equivalents as set forth in the claims.

What is claimed is:

1. A method, and systems based on the said method, which 1) utilize renewable energy, or any energy the production of which emits little or no carbon dioxide (CO₂), or the syngas produced from the said method, for generating electricity that powers the said systems; 2) convert CO₂, sustained with the said generated electricity, into methane which a) does not ecologically raise CO₂ level in the atmosphere when consumed; b) can be produced in large volume hence presents a new and CO₂-neutral natural gas resource and a replacement of, or an addition to, other natural gas resources and fossil fuels; 3) generate oxygen as byproduct, comprise 1) generating electricity, which powers the said system, from renewable energy or through the use of the generated syngas mainly composed of the said generated methane; 2) providing heating source to support hydrogen generation and/or CO₂ feed-in; 3) providing external water source for hydrogen production for the said system use; 4) providing hydrogen from the external hydrogen source to the said system; 5) providing CO₂ source that accepts and preprocesses external CO₂ as feedstock; 6) providing a set of water electrolyzers that electrolyze water for hydrogen production; 7) providing a set of Sabatier reactors that convert the CO₂ feedstock and hydrogen into methane; 8) providing a set of gas separators that separate gases after CO₂-to-methane reaction; 9) outputting gas mixture of majorly methane and some hydrogen for commercial use or storage; 10) outputting oxygen for commercial use or storage; 11) recycling water to the said water electrolyzer as feedstock; 12) recycling CO₂, H₂ and the residual mixture of CO₂, H₂, water and methane to the said Sabatier reactor; 13) recycling/reusing the heat generated at the said Sabatier reactor to the said water electrolyzer and/or to the said CO₂ acceptor and/or optionally to an alternator generating electricity for the said system use.

2. The said electricity in claim 1 is generated with no or little CO₂ emission in comparison with fossil-fuel burning generated power.

3. The said renewable energy in claim 1 includes, but is not limited to, wind, solar, hydro, nuclear, tide waves and the combination of any of them.

4. The said external hydrogen source in claim 1 provides hydrogen that is generated with no or little of CO₂ emission. It preferably is the product of water electrolysis.

5. The said CO₂ source in claim 1 accepts CO₂ that is collected from the atmosphere or from any sites where CO₂ is produced.

6. The said Sabatier reactor in claim 1 runs with catalysts and is most efficient around 300 degrees Celsius converting CO₂ and hydrogen to methane and water.

7. The said systems in claim 1 are scalable in a broad range of sizes and capabilities: can be used in regional area or for a building only.

8. The said systems in claim 1 have an efficiency ranging from 10% to 95%.
